(19) United States
(12) Patent Application Publication
(10) Pub. No.: US 2026/0182861 A1
(43) Pub. Date: Jul. 2, 2026

(54) LUNG FUNCTION MEASUREMENT ARRANGEMENT

(71) Applicant: Mincor AB, Stockholm (SE)

(72) Inventor: Andras GEDEON, Stockholm (SE)

(73) Assignee: Mincor AB, Stockholm (SE)

(21) Appl. No.: 19/129,941

(22) PCT Filed: Sep. 8, 2023
(86) PCT No.: PCT/EP2023/074762

§ 371 Date: May 14, 2025

(30) Foreign Application Priority Data
Nov. 25, 2022 (SE) 2251373-3

(51) Int. Cl. A61B 5/091; A61B 5/00; A61B 5/026

(52) U.S. Cl. CPC A61B 5/091; A61B 5/026; A61B 5/7271; A61B 5/7405; A61B 5/742; A61B 5/7455

(57) ABSTRACT

A measurement arrangement for measuring carbon dioxide partial pressure of exhaled air from a person includes a calculation unit, a carbon dioxide analyser device, and a gas conduit assembly. A perturbation volume member is provided, defining a predetermined known perturbation volume, and configured to be selectively connected and disconnected to a person such that both inspiratory and expiratory air flows can pass either through the perturbation volume member or not, thereby introducing a variation of the determined end-tidal carbon dioxide. The perturbation volume member is either a gas conduit, or a perturbation space conduit which is configured to be connected to and disconnected from the gas conduit. The calculation unit is configured to calculate the effective pulmonary blood flow (EPBF) and the effective lung volume (ELV) based upon these induced variations of the determined end-tidal carbon dioxide partial pressure, the known perturbation volume, and a respiratory rate.

### TECHNICAL FIELD

The present disclosure relates to a lung function measurement arrangement for measuring carbon dioxide partial pressure of exhaled air from a person. In particular, the disclosure relates to a measurement arrangement configured to apply the so-called differential Fick method to the carbon dioxide balance in the lung for assessing the effective lung function in a non-invasive way. Several practical ways have been devised for implementing this technique for patients on mechanical ventilation.

### BACKGROUND

The Fick's principle is a cornerstone of the physiology of gas exchange in the lungs and states that the amount of carbon dioxide gas exhaled through breathing M (exhaled) is the difference between the amount brought into the lung by the venous blood flow Mv, and the amount leaving the lung by the arterial blood flow Ma.

$$M ( exhaled ) = Mv - Ma \qquad ( 1 )$$

Because of the large body stores of carbon dioxide, any short (typically less than 30 s) perturbation of this gas exchange balance will not affect the venous inflow which therefore can be assumed to be constant under such conditions. As a result, Fick's principle for carbon dioxide can be formulated as a relation between the change in the amount of carbon dioxide eliminated through breathing and the change of the arterial blood carbon dioxide content.

$$Change in M ( exhaled ) = - Change in Ma \qquad ( 2 )$$

And since the arterial blood content of carbon dioxide is proportional to the partial pressure Pa through a constant S we get:

$$CO 2 Flow ( perturbed ) - CO 2 Flow ( equilibrium ) = EPBF \times ( Pa ( equilibrium ) - Pa ( perturbed ) ) \times S \qquad ( 3 )$$

In equation (3), CO2Flow is the amount CO2 exhaled (l/min) at equilibrium and shortly after the perturbation, respectively. EPBF is the effective pulmonary blood flow in l/min, that is the blood flow through the lungs that participate in the CO2 elimination process. Pa is the arterial CO2 partial pressure at equilibrium and shortly after the perturbation, respectively, and S is a constant relating the CO2 partial pressure in blood to the CO2 content of the blood.

For simplicity we can denote the differences in equation (3) as deltaFlow and deltaPa and then equation (3) can be written:

$$EPBF = deltaFlow / ( S \times deltaPa ) \qquad ( 4 )$$

Since small changes in Pa are closely reproduced by changes in the so-called end tidal carbon dioxide value Pet, equation (4) becomes:

$$EPBF = deltaFlow / ( S \times deltaPet ) \qquad ( 5 )$$

where Pet is the partial pressure of carbon dioxide at the very end of the expiration where the gas has been in close equilibrium with the arterial blood. Equation (5) is the differential Fick equation for CO2. It is valid irrespective of how, or how much, the equilibrium is perturbed. As a result, many ways to utilize equation (5) have been conceived.

A change of the minute ventilation was used when the method first was introduced.

Different degrees of rebreathing, breath holding and changing the respiratory rate, the so-called capnodynamic therapy, have all been successfully demonstrated. This is disclosed e.g. in following US-patents.

U.S. Pat. No. 6,302,851 relates to a method and a device for determining pulmonary blood flow (Qp), cardiac output (CO(Fick)) and the lung volume of effective gas exchange (ELV) from breath-by-breath measurements of the tidal exhaled CO2 elimination V (litre/min) and the end tidal CO2 concentration P (%) using the differential Fick method. The measurements are made during steady state ventilation and when the CO2 balance in the lungs changes subsequent to a perturbation of the gas exchange conditions. According to the method, a short breath hold is used to implement such a perturbation. V and P were measured in patients on mechanical ventilation. When the end tidal CO2 values were stable, the end inspiratory pause of a single breath was prolonged 3 seconds as compared to the normal ventilation pattern. From the changes induced in P and V, Qp, CO(Fick) and ELV are obtained. Thus, with a single breath perturbation, the differential Fick method can yield cardiopulmonary information using 2-3 breaths only and with a minimum of interference with the patient. Complete data analysis results in multiple determinations of the Qp and ELV values which improve the attainable precision.

U.S. Pat. No. 6,955,651 relates to methods for estimating the volume of the carbon dioxide stores of an individual's respiratory tract that include determining a carbon dioxide store volume at which a correlation between corresponding signals of carbon dioxide elimination and an indicator of the content of carbon dioxide in blood of the individual is optimized.

U.S. Pat. No. 7,699,788 relates to methods for noninvasively measuring, or estimating, functional residual capacity or effective lung volume include obtaining carbon dioxide concentration and flow measurements at or near the mouth of a subject.

U.S. Pat. No. 11,045,105 relates to a method for determination of cardiac output or EPBF of a mechanically ventilated subject. The method comprises the steps of introducing a change in the effective ventilation of the subject, measuring expiratory flow and CO2 during a sequence of analyzed breaths during which the effective ventilation of the subject varies, and determining the cardiac output or EPBF of the subject using the flow and CO2 measurements.

A suitable method for measuring the carbon dioxide partial pressure is colorimetric capnometry, i.e. as disclosed in U.S. Pat. No. 10,175,254, that relates to colorimetric carbon dioxide detection and measurement systems that include a gas conduit, a colorimetric indicator adapted to exhibit a color change in response to exposure to carbon dioxide gas.

When using a rebreathing method, the perturbation has not been controlled, only the resulting changes have been measured. However, the measurement of deltaFlow in equation (5) requires an accurate (better than 5% of reading), fast response (better than 0.1 s) CO2 analyzer and flowmeter because the CO2Flow and thus deltaFlow must be obtained by integration over time of the product of the instantaneous values of flow and carbon dioxide concentration. This means that errors may enter due to lack of synchronicity of the signals as well as errors in the flow and concentration data themselves.

Nevertheless, in high-end modern respirators, and anesthesia machines, deltaFlow can be measured with clinically acceptable accuracy but the sophisticated instrumentation involved would be prohibitive for a device that is to be used for diagnostic purposes during spontaneous breathing.

Because of this, the differential Fick method has until now only been used for patients on mechanical ventilation as part of vital signs monitoring. If the method could be applied also during spontaneous breathing it could help assess several diseases of the lung as well as detect and quantify pulmonary embolism, a life-threatening condition that results in reduced effective pulmonary blood flow EPBF.

The object of the present invention is to achieve a simple, easy to use, inexpensive device, for assessing gas exchange impairment in the lung, particularly during spontaneous breathing, using the differential Fick method.

### SUMMARY

The above-mentioned objects are achieved by the present invention according to the independent claim.

Preferred embodiments are set forth in the dependent claims.

The purpose of the present invention is to eliminate the need for measuring CO2Flow as described above and thus the associated complexity, costs and sources of error. It is thereby achieved a simple yet accurate measurement arrangement particularly suitable for diagnostics on spontaneous breathing subjects.

### BRIEF DESCRIPTION OF THE DRAWINGS

FIG. 1A is a graph illustrating a typical partial pressure vs volume relationship in a single expiration for a person breathing through an embodiment of a measurement arrangement according to the present invention shown in FIG. 1B.

FIG. 1B is a schematic illustration of an embodiment of a measurement arrangement according to the present invention.

FIG. 1C is a schematic illustration of the embodiment shown in FIG. 1B, in a disconnected stage of the measurement performed by the arrangement.

FIG. 1D is a schematic illustration of another embodiment of a measurement arrangement according to the present invention.

FIG. 2 is a graph illustrating two different cases of the graph of FIG. 1A, for a healthy and for a diseased lung. Also shown are the corresponding mean partial pressures of carbon dioxide in the last 0.1 l portion of the expired volume.

FIG. 3 is a graph illustrating a typical variation of Pet when, in the case shown at 40 s, the conduit 18 is disconnected from conduit 14. It is also shown how the quantities dPet and deltaPet are determined from the graph.

### DETAILED DESCRIPTION

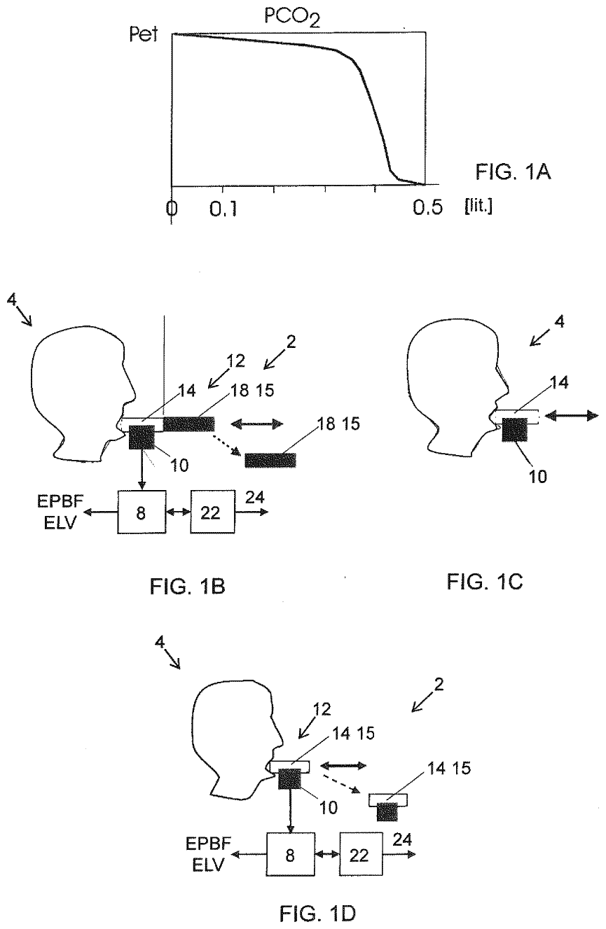

The measurement arrangement will now be described in detail with references in particular to FIG. 1A-1D. Throughout the figures the same, or similar, items have the same reference signs. Moreover, the items and the figures are not necessarily to scale, emphasis instead being placed upon illustrating the principles of the invention.

FIGS. 1B-1D illustrate exemplary and schematic set-ups of the measurement arrangement according to the present invention. Although FIGS. 1B-1D show connection to the mouth only, any embodiment connecting to the nose only or to both the mouth and the nose are possible according to the invention

A measurement arrangement 2 is provided for measuring carbon dioxide partial pressure of exhaled air from a person 4. The arrangement comprises a calculation unit 8, a carbon dioxide analyser device 10, and a gas conduit assembly 12.

The gas conduit assembly 12 comprises a gas conduit 14 configured to lead air flow to and from a person during inhalation and exhalation of the person 4. The gas conduit assembly 12 is provided with a connection member structured to connect the carbon dioxide analyser device 10 to the gas conduit 14 such that said carbon dioxide analyser device 10 is enabled to measure the carbon dioxide partial pressure of the exhaled air, and to determine the end expiratory carbon dioxide partial pressure of each breath in dependence of the measured carbon dioxide partial pressure, and of the respiratory rate.

The measurement arrangement 2 comprises a perturbation volume member 15 defining a predetermined known perturbation volume. The perturbation volume member 15 is configured to be selectively connected and disconnected to the person such that both inspiratory and expiratory air flows can pass either through the perturbation volume member 15 or not, thereby introducing a variation of the determined end-tidal carbon dioxide.

The perturbation volume member 15 being either the gas conduit 14, which is illustrated in FIG. 1D, or being a perturbation space conduit 18 which is configured to be connected to and disconnected from said gas conduit 14. This embodiment is illustrated in FIGS. 1B and 1C.

The calculation unit 8 is configured to calculate the effective pulmonary blood flow (EPBF) and the effective lung volume (ELV) based upon these induced variations of the determined end-tidal carbon dioxide partial pressure, the known perturbation volume, and the respiratory rate. The calculation unit 8 has the necessary calculation capabilities to perform the calculations of EPBF and ELV, and is e.g. a laptop computer, a tablet computer, or any type of computer provided with input means to receive the signals from the carbon dioxide analyser device 10.

According to an embodiment, the perturbation volume is in the range of 10-30%, more preferably 15-25%, and even more preferably approximately 20% of an estimated tidal volume of the person.

Preferably, the perturbation volume is estimated proportional to the person's body weight.

In a further embodiment, perturbation volume member is manually disconnected at a point of time during said person's exhalation and is connected at a point of time during said person's inspiration. The manual operation may be performed either by the person being subject to the measurement, or by a medical staff.

Preferably, the gas conduit 14 and the perturbation space conduit 18 both have a tubular shape.

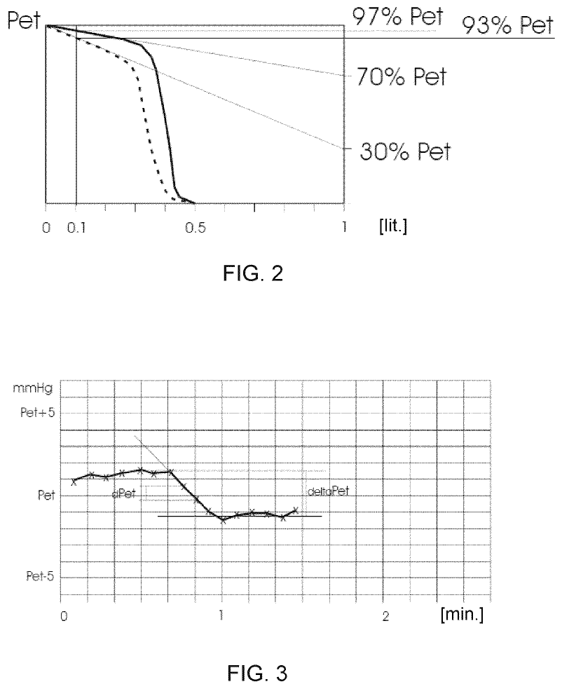

According to still another embodiment, the changes in the end expiratory carbon dioxide partial pressure when selectively connecting or disconnecting the perturbation volume member are analysed to determine EPBF and ELV according to:

$$EPBF = 0.975 \times Pet \times Vd \times RR / ( S \times deltaPet )$$

$$ELV = 0.975 \times Pet \times Vd / dPet$$

where:
Pet is the end tidal carbon dioxide partial pressure, i.e. the partial pressure at the very end of the expiration where the gas has been in close equilibrium with the arterial blood;
Vd is the volume in liter of the perturbation volume member 15;
RR is number of person's breathes per minute;
S is a constant relating the CO2 partial pressure in blood to the CO2 content of the blood; deltaPet is the difference between Pet in equilibrium before perturbation is applied and the Pet value when equilibrium is reached again after the perturbation (see FIG. 3), and dPet is the change in Pet for each breath immediately following the application of the perturbation (see FIG. 3).

The equations for calculating EPBF and ELV will be discussed in detail below.

In another embodiment illustrated in FIGS. 1B and 1D, the arrangement is provided with a pacing member 22 configured to generate a tactile, audible, and/or visible signal 24 at a predetermined and selectable rate to guide the person 4 to keep a steady respiratory rate. Advantageously, the pacing member 22 is configured to generate an audible signal 24 having different sounds for inspiration and expiration, respectively. Preferably, the pacing member 22 is controlled by the calculation unit 8.

FIG. 1A is a graph illustrating the carbon dioxide partial pressure variation in an imaged tube having a volume of 0.5 l and as shown in FIG. 1B at the end of an exhalation for a person having a tidal breath of 0.5 l, that is just enough to fill the tube. The CO2 partial pressure varies along the imagined tube with the partial pressure closest to the patient being, by definition, equal to the Pet value.

FIG. 1B shows an embodiment where the patient breathes not only through conduit 14 with the CO2 analyzer device but also through a perturbation space conduit 18, thus being the perturbation volume member 15, that in this exemplary set-up has a volume of 0.1 l.

FIG. 1C shows breathing when this added perturbation space conduit 18 has been removed. In FIG. 1B, the disconnection of the perturbation space conduit 18 is schematically indicated by a dashed arrow.

FIG. 1D shows another embodiment where the patient breathes only through conduit 14 with the CO2 analyzer device, thus being the perturbation volume member 15. The dashed arrow schematically indicates the removal of the perturbation volume member 15, i.e. the conduit 14.

The key aspects of the measurement arrangement according to the present invention, are that the perturbation volume member 15 has a precisely known volume and that this volume is small enough only to contain the last portion of the exhaled gas having a partial pressure very close to the Pet value.

In the set-up shown in FIG. 1B, this volume is 0.1 l. In general, i.e. also applicable in the embodiment where the perturbation volume member 15 is embodied by the conduit 14, if the volume of the perturbation volume member 15 is Vd litre, then the amount of CO2 in the tube will be (Pet×Vd) litre and if the patient is breathing RR times per minute then the amount of CO2 inflow from the perturbation space is (Pet×Vd×RR) l/min. Then:

$$deltaFlow = Pet \times Vd \times RR ,$$

and equation (5) becomes:

$$EPBF = Pet \times Vd \times RR / ( S \times deltaPet ) \qquad ( 6 )$$

If equilibrium is present without perturbation conduit 18 attached to conduit (14), as in FIG. 1C, then connecting perturbation conduit 18 will perturb the system and increase Pet and equation (6) would still be valid.

It should be noted that, according to equation (6), there is no longer need for gas flow measurements and both the nominator and the denominator has only Pet as measured parameter. Because of this, the CO2 analyser device does not need to be accurately calibrated since any error in the calibration will produce the same factor both in the nominator and the denominator and thus cancel out.

Furthermore, because Pet is measured, by definition, on the flat part of the CO2 concentration curve (see FIG. 1A), the carbon dioxide analyser device 10 does not need to have a very fast response time.

However, as shown in FIG. 1A, the CO2 partial pressure (shown at the Y-axis) versus volume (shown at the X-axis) curve has a minor slope at the end of expiration which means that the partial pressure decreases in the perturbation space away from the patient and will always be on average somewhat lower than the Pet value.

FIG. 2 illustrates how different slopes correspond to different average partial pressures in a perturbation space of 0.1 l at a tidal volume of 0.5 l. The slope of the CO2 partial pressure as a function of the exhaled volume has been studied extensively in both healthy persons, and in persons having different diseases.

In FIG. 2, the slope giving a 30% reduction in Pet at 1 litre, corresponds to essentially healthy lungs while the dashed line of 70% reduction corresponds to severe cases of emphysema, a condition that produces the largest slopes.

As is shown, the average partial pressure of CO2 is 0.985×Pet for the healthy case while in the emphysema case the average partial pressure is reduced to 0.965×Pet.

Thus, to account, to the first order, for the various possible slopes of the CO2 partial pressure curve, Pet in equation (6) can be corrected with a factor of 0.975+/−0.01 to give a final formula:

$$EPBF = 0.975 \times Pet \times Vd \times RR / ( S \times deltaPet ) \qquad ( 7 )$$

If the effective lung volume participating in the gas exchange is denoted ELV then removing or attaching the perturbation space conduit, means removing/adding the carbon dioxide amount of 0.975×Pet×Vd on each subsequent breath.

If this results in a partial pressure drop/increase of dPet for each breath, then

$$ELV = 0.975 \times Pet \times Vd / dPet \qquad ( 8 )$$

The expression for ELV is again error compensating just as is the expression for EPBF.

FIG. 2 demonstrates the importance of choosing as small perturbation space volume as possible. In this way the correction factor in equations (7) and (8) can be minimized. However, the perturbation space volume must still be large enough to produce a significant enough change in Pet as compared to the noise overlaying the measured Pet data, otherwise the precision (reproducibility) of the measured EPBF and ELV will be jeopardized. A perturbation space volume set to about 20% of the tidal volume is a good compromise between these conflicting goals.

As a rule of thumb the tidal volume of a person can be calculated as 0.007 l/kg bodyweight, so a 70 kg person will have approximately a tidal volume of 0.49 l, and should be subjected to a perturbation space volume of about 0.1 l as illustrated in FIG. 1B This also means that perturbation space volumes in the range 75 ml-150 ml would adequately cover the normal tidal volume range.

FIG. 3 shows a typical recording from a healthy individual before and shortly after the removal of the perturbation space tube and shows how the denominators of equations (7) and (8) are determined from the recording. In FIG. 3, the Y-axis represents the change in the equilibrium Pet in mmHg and the X-axis represents time in minutes.

According to the above, the present invention involves a precisely known volume, small enough only to contain the end-tidal portion of the exhaled gas, to be connected to or detached from a spontaneously breathing person in gas exchange equilibrium, thereby introducing a perturbation of the carbon dioxide inflow/outflow to/from the lungs. The resulting variation of the end-tidal carbon dioxide partial pressure allows the calculation of the effective pulmonary blood flow (EPBF) and the effective lung volume (ELV).

In order to help keep the breathing rate RR as constant as possible the patient can be asked to breath in synchrony with rising and falling sound signals (at the natural respiratory rate of the patient) corresponding to inhalation and exhalation.

In one preferred embodiment the perturbation space volume is formed as a tube conduit with known inner diameter and length being connected during the steady state and disconnected during an expiration to induce the required perturbation in the gas exchange. The perturbation space volume is suitably chosen as 20% of the tidal volume or approximately as 1.4 ml/kg body weight.

The CO2 analyzer device to be used can be of any of the standard IR type (i.e. EMMA, Masimo Corporation US) but could also equally well employ a more inexpensive technology such as colorimetric capnometry.

The measurement arrangement according to the present invention uses only a CO2 analyzer device and is therefore particularly advantageous for diagnostic measurements on spontaneous breathing subjects. Nevertheless, it can equally well be used for monitoring patients on mechanical ventilation even if the ventilator lacks flow measuring capability.

The present invention is not limited to the above-described preferred embodiments. Various alternatives, modifications and equivalents may be used. Therefore, the above embodiments should not be taken as limiting the scope of the invention, which is defined by the appending claims.

1. A measurement arrangement for measuring carbon dioxide partial pressure of exhaled air from a person, the arrangement comprises a calculation unit, a carbon dioxide analyser device, and a gas conduit assembly, and that the gas conduit assembly comprises a gas conduit configured to lead air flow to and from a person during inhalation and exhalation of the person, wherein said gas conduit assembly is provided with a connection member structured to connect said carbon dioxide analyser device to said gas conduit such that said carbon dioxide analyser device is enabled to measure the carbon dioxide partial pressure of the exhaled air, and to determine the end expiratory carbon dioxide partial pressure of each breath in dependence of the measured carbon dioxide partial pressure, and of the respiratory rate, wherein the measurement arrangement comprises a perturbation volume member defining a predetermined known perturbation volume, wherein said perturbation volume member is configured to be selectively connected and disconnected to the person such that both inspiratory and expiratory air flows can pass either through said perturbation volume member or not, thereby introducing a variation of the determined end-tidal carbon dioxide, and that said perturbation volume member being either said gas conduit, or being a perturbation space conduit which is configured to be connected to and disconnected from said gas conduit, and wherein said calculation unit is configured to calculate the effective pulmonary blood flow (EPBF) and the effective lung volume (ELV) based upon these induced variations of the determined end-tidal carbon dioxide partial pressure, the known perturbation volume, and the respiratory rate.

2. The arrangement according to claim 1, wherein the perturbation volume is in the range of 10-30%, more preferably 15-25%, and even more preferably approximately 20% of an estimated tidal volume of the person.

3. The arrangement according to claim 1, wherein the perturbation volume is estimated proportional to the person's body weight.

4. The arrangement according to claim 1, wherein said perturbation volume member is manually disconnected at a point of time during said person's exhalation and is connected at a point of time during said person's inspiration.

5. The arrangement according to claim 1, wherein the changes in the end expiratory carbon dioxide partial pressure when selectively connecting and disconnecting the perturbation volume member are analysed to determine EPBF and ELV according to:
$$EPBF = 0.975 \times Pet \times Vd \times RR / ( S \times deltaPet )$$
$$ELV = 0.975 \times Pet \times Vd / dPet$$
where:
Pet is the end tidal carbon dioxide partial pressure at the end of the expiration where the gas has been in close equilibrium with the arterial blood;
Vd is the volume in liter of the perturbation volume member;
RR is number of person's breathes per minute;
S is a constant relating the CO2 partial pressure in blood to the CO2 content of the blood;
deltaPet is the difference between Pet in equilibrium before perturbation is applied and the Pet value when equilibrium is reached again after the perturbation, and
dPet is the change in Pet for each breath immediately following the application of the perturbation.

6. The arrangement according to claim 1, comprising a pacing member configured to generate a tactile, audible, and/or visible signal at a predetermined and selectable rate to guide the person to keep a steady respiratory rate.

7. The arrangement according to claim 6, wherein the pacing member is configured to generate an audible signal having different sounds for inspiration and expiration, respectively.

8. The arrangement according to claim 1, wherein the gas conduit, and the perturbation space conduit, both have a tubular shape.
